(19) United States
(12) Patent Application Publication
(10) Pub. No.: US 2024/0404690 A1
(43) Pub. Date: Dec. 5, 2024

(54) SYSTEMS AND METHODS FOR MAINTENANCE SERVICES

(71) Applicant: KONINKLIJKE PHILIPS N.V., EINDHOVEN (NL)

(72) Inventors: Tiblets Zeray Demewez, EINDHOVEN (NL); Mauro Barbieri, EINDHOVEN (NL); Qi GAO, EINDHOVEN (NL); Milosh Stolikj, EINDHOVEN (NL)

(21) Appl. No.: 18/692,352

(22) PCT Filed: Sep. 16, 2022
(86) PCT No.: PCT/EP2022/075715

§ 371 Date: Mar. 15, 2024

Related U.S. Application Data
(60) Provisional application No. 63/247,393, filed on Sep. 23, 2021.

(51) Int. Cl. G16H 40/40; G06Q 10/0631; G06Q 10/20

(52) U.S. Cl. CPC G16H 40/40; G06Q 10/063114; G06Q 10/20

(57) ABSTRACT

A non-transitory computer readable medium (107, 127) stores data related to medical imaging devices (120) generated from log files (132) from the medical imaging devices; and instructions readable and executable by at least one electronic processor (101, 113) to: retrieve, based on a query received for a current service case, log data from the log files of the medical imaging device for which the current service case is to be performed including at least operational data of the medical imaging device related to the current service case to be performed; generate, from the retrieved log data, at least a subset of the operational data for the current service case to be provided to a maintenance provider; and provide, on a display device (105) of an electronic processing device (102) operable by the maintenance provider, a user interface (UI) (140) displaying information about the current service case.

### FIELD

The following relates generally to the medical device maintenance arts, predictive maintenance arts, maintenance part ordering and recommendation arts, and related arts.

### BACKGROUND

Customer services for medical systems can require reactive, proactive, and predictive maintenance services. Because these services rely heavily on data, availability of a reliable infrastructure that acquires, processes and stores data from medical systems and other sources is crucial. When there is a problem with a medical system, customers can report the issue to the customer service center via various channels (e.g., by phone, customer portal or mobile application). Then, service engineers (SEs-which can include remote SEs (RSEs), remote monitoring engineers (RMEs), and field SEs (FSEs)) can perform system diagnostics to identify the root-cause of the problem and resolve the issue. Both activities may be performed remotely or on-site, by different SEs. In addition to reactive maintenance as described above, proactive, and predictive diagnostic models are built to identify failure of a system or a part of a system. The output of the diagnostic models is communicated to RSEs or RMEs, in a form of alert, using a service delivery platform. The RSEs or RMEs act on the alerts to provide maintenance services.

Medical device vendors may provide maintenance services under contract with customers for various medical systems such as image-guided therapy (iGT) systems, computed tomography (CT) systems, magnetic resonance (MR) systems, Ultrasound (US) systems, and so forth. However, medical device vendors may also outsource some maintenance cases to Third-party Maintenance Providers (TMPs), or the customer may directly contract with a TMP to provide maintenance services. In either case, the vendor typically continues monitoring the systems and forwards alerts to the customer and/or the TMP.

Issues from medical systems are identified in two ways. The first way is when customers observe a system malfunction during utilization and report the issue to customer services. The second way is using proactive and preventive diagnostic models. Both cases utilize device data to investigate the issue and find a solution. Medical systems generate device (i.e., log) data that provide information regarding the usage of the system. These files are processed and readily available for troubleshooting, root cause analysis as well as building proactive and predictive diagnostic models. These models are run against the log data and the outcome, in the form of an alert, is displayed within a service delivery platform. Every alert is reviewed by an RSE and followed by case creation when applicable. Case creation triggers a service work order that initiates resolution of an alert.

The following discloses certain improvements to overcome these problems and others.

### SUMMARY

In one aspect, a non-transitory computer readable medium stores data related to medical imaging devices generated from log files from the medical imaging devices; and instructions readable and executable by at least one electronic processor to: retrieve, based on a query received for a current service case, log data from the log files of the medical imaging device for which the current service case is to be performed including at least operational data of the medical imaging device related to the current service case to be performed; generate, from the retrieved log data, at least a subset of the operational data for the current service case to be provided to a maintenance provider; and provide, on a display device of an electronic processing device operable by the maintenance provider, a user interface (UI) displaying information about the current service case.

In another aspect, a non-transitory computer readable medium stores data related to medical imaging devices generated from log files from the medical imaging devices; and instructions readable and executable by at least one electronic processor to: retrieve, based on a query received for a current service case, log data from the log files of the medical imaging device for which the current service case is to be performed including at least operational data of the medical imaging device related to the current service case to be performed; generate, from the retrieved log data, at least a subset of the operational data for the current service case to be provided to a maintenance provider; and provide, on a display device of an electronic processing device operable by the maintenance provider to perform the current service case, a UI displaying information about the current service case and presenting a dialog for ordering parts for the medical imaging device for which the current service case is to be performed.

In another aspect, a non-transitory computer readable medium stores data related to qualifications of a plurality of maintenance providers; data related to medical imaging devices generated from log files from the medical imaging devices; instructions readable and executable by at least one electronic processor to: retrieve, based on a query received for a current service case, log data from the log files of the medical imaging device for which the current service case is to be performed including at least operational data of the medical imaging device related to the current service case to be performed; generate, from the retrieved log data, at least a subset of the operational data for the current service case to be provided to a maintenance provider; retrieve the qualifications of maintenance providers of the plurality of maintenance providers; select a maintenance provider from the plurality of maintenance providers to perform the current service case based on the retrieved log data and the retrieved qualifications; and provide, on a display device of an electronic processing device operable by the selected maintenance provider, a UI displaying information about the current service case and presenting a dialog for ordering parts for the medical imaging device for which the current service case is to be performed.

One advantage resides in minimizing time to resolve a service issue for a medical device.

Another advantage resides in anonymizing shared data between a medical device vendor and a TMP for the TMP to perform a service case for a medical device.

Another advantage resides in ensuring that customer data is not compromised while a TMP performs a maintenance service for a device owned or operated by the customer.

Another advantage resides in facilitating payments between a customer and a TMP after the TMP performs a maintenance service for the customer.

A given embodiment may provide none, one, two, more, or all of the foregoing advantages, and/or may provide other advantages as will become apparent to one of ordinary skill in the art upon reading and understanding the present disclosure.

### BRIEF DESCRIPTION OF THE DRAWINGS

The disclosure may take form in various components and arrangements of components, and in various steps and arrangements of steps. The drawings are only for purposes of illustrating the preferred embodiments and are not to be construed as limiting the disclosure.

FIG. 1 diagrammatically illustrates an illustrative system for determining a root cause and at least one recommended service action for servicing a medical device in accordance with the present disclosure.

FIGS. 2 and 3 show exemplary flow chart operations of the system of FIG. 1.

### DETAILED DESCRIPTION

In the medical imaging field, medical device vendors provide support to customers in part by receiving machine log data uploaded from the imaging devices in the field. In one support mode, various failure prediction models are automatically run on the machine log data. Alerts generated by these models are forwarded to a Remote Service Engineer (RSE) based at the vendor, who reviews the alert and contacts the customer if maintenance is needed. Additionally or alternatively, the alert may be sent directly to the customer. This provides a mechanism for initiating preventative service. In another support mode, an RSE receives calls from customers presently experiencing imaging device problems.

In either support mode, a service case is opened at the vendor side when preventative or repair work is needed. The service case may be handled in-house by a Field Service Engineer (FSE) working for the vendor, or may be handled by a Third-party Maintenance Provider (TMP).

A fleet of hundreds, thousands, or more deployed medical imaging devices generates a large number of reported issues and alerts that cover a broad range of issues as well as resolution approaches. To minimize the amount of time required to resolve an issue, involving a TMP may be beneficial. However, the involvement of TMP may raise issues as to the extent to which data regarding the medical imaging device and its use that may be collected by the medical imaging device vendor to provide proactive maintenance alerts and similar services to the customer should be shared with the TMP. This applies also when customers contract directly with a TMP to perform maintenance tasks, but the imaging device vendor continues to provide other services such as proactive maintenance alerts to the customer. As a further issue, if the medical device vendor (sub-) contracts with TMPs to provide maintenance to customers, then the medical device vendor is responsible for selecting qualified TMPs for specific maintenance cases. Given the wide diversity of medical imaging devices, this selection can be challenging.

The following relates to a situation in which a TMP handles the case, and discloses a mechanism for the TMP to be selected and/or for the TMP to receive secure access to the machine log data stored on the vendor's server.

Upon opening of a service case for which a TMP is to be assigned, a database of service provider qualification data is analyzed to recommend a TMP who is qualified to perform the service. The recommendation approach accounts for factors such as the TMP's experience, likelihood of successfully resolving the service case, and cost estimate.

The disclosed system also collects machine log data that is relevant for the service case. These may include alerts issued by failure prediction models, log content generated proximate to the time of the alert or customer call and/or related to the malfunctioning system or subsystem, results of automated root cause detection patterns run on the log data, imaging device model and configuration data, and so forth. The collected data are anonymized to ensure HIPAA compliance, and may also be filtered to remove proprietary information that is owned by the medical device vendor. For example, an alert issued by a proprietary failure prediction model may include supporting information on which basis the model issued the alert, which is presented to an RSE reviewing the alert. However, if that supporting information could be used to reverse-engineer the proprietary failure prediction model then only the alert and possibly a subset of the supporting information may be provided to the TMP. The collected and anonymized/filtered relevant machine log data are then transferred to the TMP via a secure Internet connection or other secure communication channel.

During the TMP service call, any parts ordered by the TMP may, in some embodiments, be required by the parts ordering system to be accompanied by an associated error code. The part information and error code, optionally along with other information such as the model/configuration of the imaging device and the identity of the malfunctioning system or subsystem are run through a verification filter to ensure that the ordered part is appropriate. If an order for a part is unable to be automatically verified, then this may be escalated to a RSE and/or to the customer to obtain approval before the order can be placed. The information used by the verification filter, along with whether the order is ultimately placed or withdrawn, may also optionally be used as training data to dynamically train or tune the verification filter.

After the service call is complete, the system may perform automated remote tests or SEs may perform on-device tests to confirm the problem has been resolved. If these tests indicate the problem is resolved, then the service case is closed. Optionally, payment to the TMP may also be conditioned on successful problem resolution.

It will be appreciated that various portions of the overall disclosed illustrative system may be usefully deployed without other portions of the overall system. For example, in the case of a TMP directly contracted by the customer, the portion of the system providing for TMP selection may be omitted. If the parts ordering system does not require an error code, then order verification based on the error code may be omitted. These are merely illustrative variants.

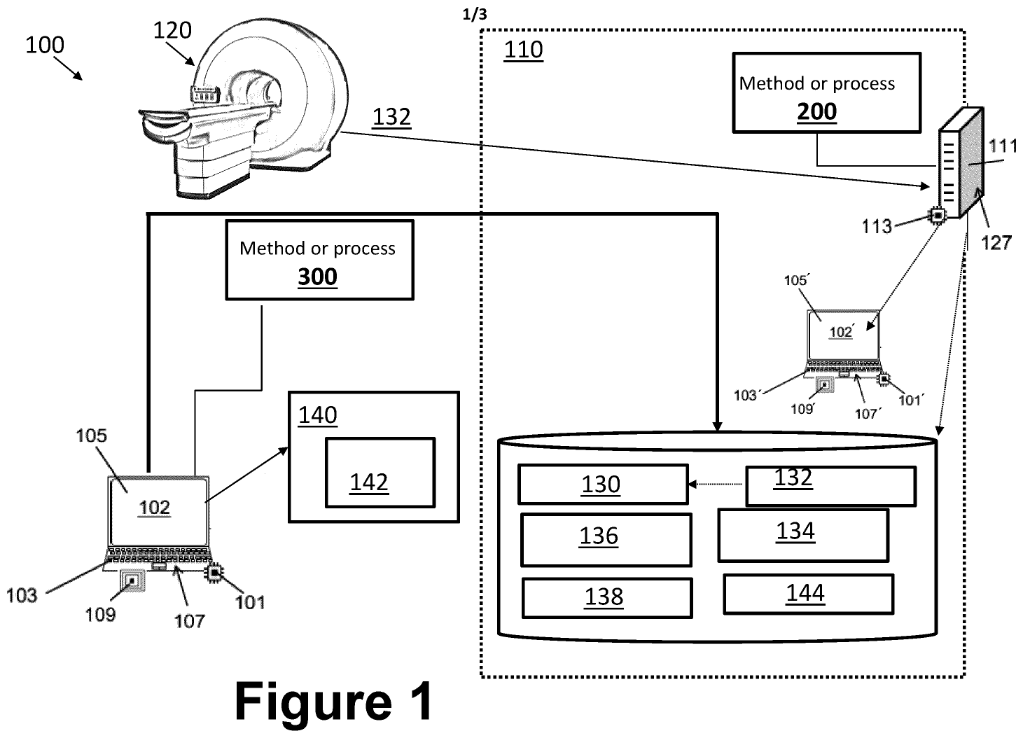

With reference to FIG. 1, an illustrative servicing support system 100 for supporting a service engineer in servicing a device 120 (e.g., a medical imaging device—also referred to as a medical device, an imaging device, imaging scanner, and variants thereof) is diagrammatically shown. By way of some non-limiting illustrative examples, the medical imaging device under service may be a magnetic resonance imaging (MRI) scanner, a computed tomography (CT) scanner, a positron emission tomography (PET) scanner, a gamma camera for performing single photon emission computed tomography (SPECT), an interventional radiology (IR) device, or so forth. (More generally, the disclosed approach can be applied in conjunction with any type of computerized device that automatically generates log data that are analyzed by predictive models to predict component failures, e.g., the approach could be applied to a commercial airliner, radiation therapy device, or so forth). As shown in FIG. 1, the servicing support system 100 includes, or is accessible by, a service device 102 that may for example be a workstation used by a TMP. The service device 102 may for example be a portable device such as a notebook computer that is carried or accessed by a TMP. The service device 102 can be a desktop computer or a personal device, such as a mobile computer system such as a laptop or smart device. In other embodiments, the service device 102 may be an imaging system controller or computer integral with or operatively connected with the imaging device undergoing service (e.g., at a medical facility). As another example, the service device 102 may be a portable computer (e.g., notebook computer, tablet computer, or so forth) carried by a TMP performing diagnosis of a fault with the imaging device and ordering of parts. In another example, the service device 102 may be the controller computer of the imaging device under service, or a computer based at the hospital. In other embodiments, the service device may be a mobile device such as a cellular telephone (cellphone) or tablet computer.

The service device 102 includes a display 105 via which alerts generated by predictive failure models are displayed, along with likely root cause and service action recommendation information as disclosed herein. The service device 102 also preferably allows the service engineer to interact with the servicing support system via at least one user input device 103 such a mouse, keyboard, or touchscreen. The service device further includes an electronic processer 101 and non-transitory storage medium 107 (internal components which are diagrammatically indicated in FIG. 1). The non-transitory storage medium 107 stores instructions which are readable and executable by the electronic processor 101 for interfacing with the servicing support system 100. The service device 102 also includes a communication interface 109 to communicate with a backend server or processing device 111, which typically implements the computational aspects of the servicing support system 100 (e.g., the server 111 has the processing power for implementing computationally complex aspects of the servicing support system 100). Such communication interfaces 109 include, for example, a wired and/or wireless Ethernet interface (e.g., in the case in which the service device 102 is a RSE workstation); or in the case in which the service device 102 is a portable FSE device the interface 109 may be a wireless Wi-Fi or 4G/5G interface or the like for connection to the Internet and/or an intranet. Some aspects of the servicing support system 100 may also be implemented by cloud processing or other remote processing (that is, the server computer 111 may be embodied as a cloud-based computing resource comprising a plurality of interconnected servers).

In illustrative FIG. 1, the servicing support system further includes a backend 110 (e.g., implemented and/or owned by the imaging device vendor or leased by the vendor from a cloud computing service provider). The backend 110 receives log data (e.g., a machine log automatically generated by the medical imaging device 120, a service log for the medical imaging device 120, and/or so forth) on a continuous or occasional basis (e.g., in some setups the imaging device 120 uploads machine log entries to the backend 110 on a daily basis). The backend processing for performing predictive fault modeling and (as disclosed herein) maintenance service analyses is performed on the backend server 111 equipped with an electronic processor 113 (diagrammatically indicated internal component). The server 111 is equipped with non-transitory storage medium 127 (internal components which are diagrammatically indicated in FIG. 1). While a single server computer is shown, it will be appreciated that the backend 110 may more generally be implemented on a single server computer, or a server cluster, or a cloud computing resource comprising ad hoc-interconnected server computers, or so forth. Furthermore, while FIG. 1 shows a single medical imaging device 120, more generally the database backend 110 will receive log data from many medical imaging devices (e.g., tens, hundreds, or more imaging devices) and performs the disclosed processing for each such medical imaging device.

In addition, the backend 110 also includes a corresponding service device 102′ operable by the RSE. As shown in FIG. 1, the service device 102′ operable by the RSE includes substantially the same component as the service device 102 (shown in FIG. 1 with prime marks, and the description of such components are not repeated here for brevity. Again, there may be any number of RSE service devices 102′.

With continuing reference to FIG. 1, the non-transitory computer readable medium 127 stores qualification data 130 related to qualifications of a plurality of maintenance providers (i.e., TMPs). The qualification data 130 can include, for example, data related to one or more of an experience level of each maintenance provider, a likelihood of success of each maintenance provider in resolving the current service case, a cost estimate for each maintenance provider, and so forth. In this regard, it should be noted that “third party maintenance provider”, i.e., “TMP”, is intended to broadly refer to any entity providing maintenance service to imaging devices (other than the vendor of those imaging devices) and for that purpose is to be given limited access to the imaging device data collected by the vendor.

The non-transitory computer readable medium 127 can also store data related to the medical imaging devices 120 generated from log files 132 from the medical imaging devices 120. The log files 132 can be transferred to the backend server 111 on a continuous or occasional basis. The data in the log files 132 can include, for example, one or more of alerts issued by one or more failure prediction models, log content generated proximate to a time of an alert, results of an automated root cause detect pattern run on the log data, a model of the medical imaging device 120, configuration data of the medical imaging device 120, and so forth.

The non-transitory computer readable medium 127 can also store one or more predictive models 134 (i.e., a failure prediction model). The predictive models 134 are configured to generate an alert 136 configured to predicting a failure of a component of the medical imaging device 120 by applying patterns to values of a set of features for the medical imaging device 120 obtained from the log files 132.

The non-transitory computer readable medium 127 can also store one or more authorized parts lists 138 for error codes associated with the medical imaging device 120 which is the subject of the current service case.

The non-transitory storage medium 127 stores instructions executable by the electronic processor 113 of the backend server 111 to perform a method 200 of providing information about a current service case for maintenance of the medical imaging device 120.

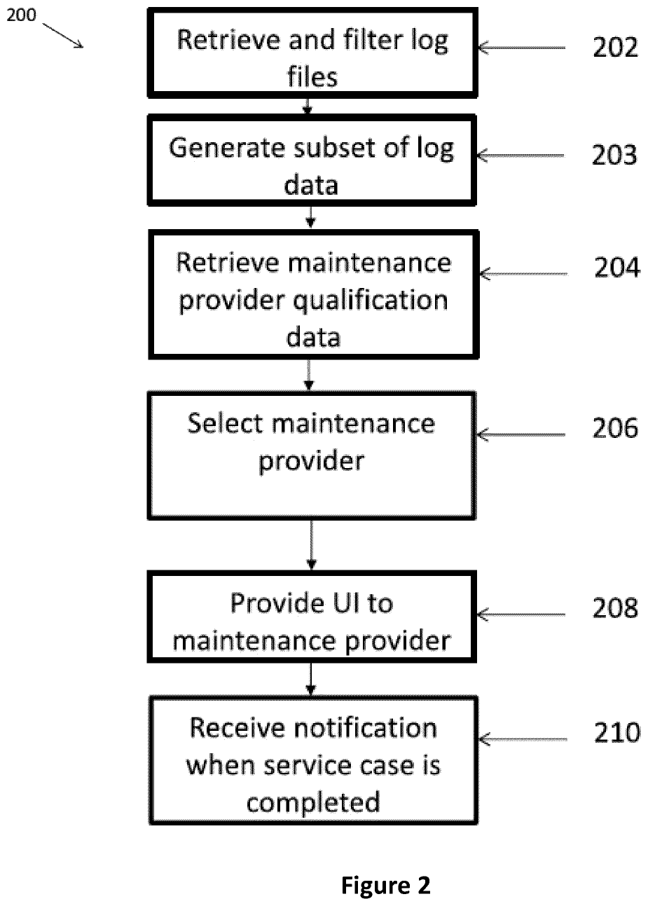

With continuing reference to FIG. 1 and further reference to FIG. 2, an illustrative embodiment of the method 200 executable by the electronic processor 113 is diagrammatically shown as a flowchart. In some examples, the method 200 may be performed at least in part by cloud processing.

To begin the method 200, at an operation 202, a query for a current service case related to the medical imaging device 120 (i.e., a “current” medical imaging device 120) can be received by the backend server 111 (operable by the vendor) from the customer of the medical device 120. From this query, log data from the log files 132 of the current medical imaging device 120 can be retrieved. In some embodiments, the retrieved log data can include at least operational data of the medical imaging device 120 related to the current service case to be performed. For example, the operational data of the medical imaging data 120 may include control parameters for the medical imaging device 120 used in recent imaging sessions, error codes recently-generated by the medical imaging device 120, sensor data generated by the medical imaging device 120 that may be relevant to the current service case, and so forth. The retrieved log data may also include other information not related to the technical operation of the medical imaging device 120, such as patient information.

At an operation 203, at least a subset of the operational data for the current service case to be provided to a maintenance provider is generated from the retrieved log data. In some embodiments, the log data from the log files 132 can be anonymized to ensure HIPAA compliance, and may also be filtered to remove proprietary information. To do so, the log data from the log files 132 can be filtered to remove proprietary information related to the medical imaging device 120. The filtering may also optionally remove information that is irrelevant to the maintenance that is to be provided by the TMP. For example, if the service call relates to a malfunction of the patient support of the medical imaging device, then log data that does not pertain to the patient support may be filtered out. The filtering operation 203 preferably retains the technical information related to the operation of the medical imaging device 120 (e.g., control parameters, error codes, sensor data, and so forth) while filtering non-technical data such as patient information.

At an optional embodiment 204, the qualification data 130 of maintenance providers of the plurality of maintenance providers can also be retrieved responsive to the query. At an operation 206, a maintenance provider from the plurality of maintenance providers is selected based on the retrieved log data and the retrieved qualifications. The operations 204, 206 may optionally be omitted, for example if the maintenance provider is not selected by the imaging device vendor (e.g., if the customer directly contracts with the maintenance provider for maintenance service) or if the maintenance provider is selected manually or by some other mechanism.

At an operation 208, a user interface (UI) 140 is provided on an electronic processing device operable by the selected maintenance provider (i.e., the service device 102). The UI 140 can display information about the current service case, and also present a graphical UI (GUI) 142 for ordering parts for the medical imaging device for which the current service case is to be performed. This may entail the maintenance provider logging onto the backend 110 using login credentials (e.g., password and/or multi-factor authentication information, et cetera) at which point the maintenance provider is recognized as the selected maintenance provider for this maintenance task and the UI 140 is presented to the maintenance provider. The displayed information can include, for example, the anonymized and filtered log data, an associated error code for the current service case, and so forth.

In some embodiments, one or more of the failure prediction models 134 are retrieved and applied to the log data from the log files 132 of the current medical imaging device 120 to generate a failure prediction based on a subset of the log data. (More generally, the failure prediction model may have been applied to the log data at some prior time, e.g., before the operations 202, 203, 204, 206, 208, and in some instances the failure prediction may have been the genesis of the maintenance task to be performed). The failure prediction, and an identification of the subset of the log data on which the failure prediction is based, can also be displayed on the UI 140. The information displayed on the UI 136 can include the failure prediction, but does not include the identification of the subset of the log data on which the failure prediction is based. This reduces the likelihood that the maintenance provider might be able to determine vendor-proprietary information such as the informational basis on which the failure prediction is based.

The maintenance provider then operates the GUI 142 to order one or more parts for the medical imaging device 120, if the maintenance provider determines such parts may resolve the maintenance problem. Responsive to the order, the backend server 111 is configured to apply a verification filter 144 comparing the ordered one or more parts with the authorized parts list 142 for the associated error code for the current service case to determine whether the one or more parts are on the authorized parts list 142 for the associated error code. If the ordered parts are on the authorized parts list 142, then the order is approved. In some examples, the verification filter 144 can be applied to one or more of a model of the medical imaging device 120, a configuration of the medical imaging device 120, and an identification of the medical imaging device 120. If the ordered parts are not on the authorized parts list 142, then a notification is transmitted from the backend server 111 or the service device 102′ operable by the RSE to the service device 102 operable by the maintenance provider.

At an operation 210, a notification is received from the service device 102 that the current service case has been completed. In response, one or more tests are performed on the current medical imaging device 120 to confirm that the current service case has been resolved. Upon determining that the current service case has been resolved, a payment is initiated to the selected maintenance provider.

### Examples

The following describes another example of the method 300. The support system 100 comprises an application within a service delivery platform that interfaces with tools that are used to provide maintenance service and relevant data sources. This application is launched when one indicates involvement of a TMP to resolve an issue reported by a customer or identified by a diagnostic model. The application ensures the selection of an appropriate TMP, and the provision of relevant information required to resolve the issue without compromising customers data ownership.

When customers report an issue, typically a troubleshooting is carried out to find the root cause and most appropriate service actions. Normally, when an issue is identified by a diagnostic model and an alert is generated, an RSE reviews the alert and creates a case when applicable. The creation of a case triggers a service work order, and the proceeded steps follow accordingly. By applying the proposed method, a customer report or an alert prompts an application that facilitates the selection of the right TMP. This can be done into two ways: (1) perform a pre-analysis operation to understand the difficulty of the issue and decide whether a root cause analysis is required before selecting a TMP; and (2) search directly a suitable TMP from a database with the corresponding service capability and other relevant information that may be required.

In the first option, the pre-analysis operation is performed if root cause analysis is carried out, and the root causes of the issue and service action is shared with the TMP without detailed data regarding the system. This is only applicable when a vendor decides to outsource the maintenance service. In the second option, following the selection of a suitable TMP and/or an available TMP (which can require a TMP bidding for the maintenance service case to provide availability schedules of individual FSEs), a rule-based logic that collects relevant information from internal systems is executed and the information is shared with the TMP. In order not to compromise customer data, a security assessment is performed to analyze the information. The security assessment investigates the data access requirements specified in the service level agreement between the vendor and the customer. When the analysis passes the security assessment, the TMP will have access to the information and can go forward with offering maintenance service. After the maintenance is carried out, the TMP sends a confirmation that signals the completion of maintenance. In addition, it sends data regarding the issue and resolution back to the vendor that assists in minimizing future parts ordered. The confirmation signal initiates a process from the vendor that validates the resolution of the issue and triggers the customer to pay the TMP for the service and closes the case. In case the issue is not resolved, feedback is given to the TMP to rework on the issue, or a new TMP is selected.

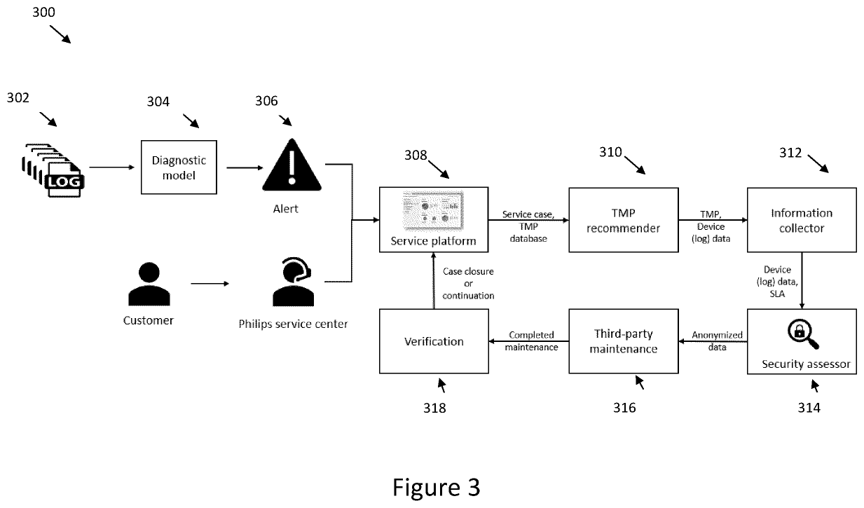

As shown in FIG. 3, log data 302 generated by the medical imaging device 120 (not shown in FIG. 3) is generated, and used to troubleshoot issues reported by customers and build diagnostic models 304. Once a customer issue is reported, an alert 306 is generated.

A new service case creation operation 308 is configured to create a service case when a problem is discovered with a medical system. The service case creation can be done programmatically, for example when a tool that monitors a medical system creates an alert that the system is malfunctioning. Service case creation can be done manually as well, for example by an RSE that regularly monitors an application in the service delivery platform to check availability of alerts that require an action. Similarly, a service case can be created manually by a support engineer when a customer calls to report a problem with a medical system.

In either case, each service case may be associated with verification tests that, if completed successfully, verify that the problem has been resolved. The service case is then a trigger to search for a suitable third-party maintenance service provider.

There are multiple ways that may trigger the creation of a new case with the new service case creation module 308. In reactive maintenance, a customer may contact the vendor to report a problem with a medical system. The customer may then use an automated tool to report the problem (e.g., customer portal or an interactive user interface as a chatbot) or may report the problem to a vendor representative (e.g., application specialist, customer support call center, etc.). In the former case, a service case is created automatically for the reported problem. In the latter case, a vendor employee creates a service case manually.

In proactive maintenance, an automated tool monitors the performance of a medical system. If the tool detects an abnormality, it creates an alert. Then, an RSE may decide to create a service case based on one of these alerts. Similarly, a service case may be created automatically based on alerts that indicate a severe problem.

The created service case may also contain a list of tests to execute in order to verify that the reported problem has been completed. Such a set of verification tests is denoted with V={v_i (s), i=1 . . . n}, where s is the medical system under maintenance, and each verification test v_i is a function that returns a binary value 0 or 1, indicating whether the verification test has failed or passed, respectively. Verification tests are preferably automated and can be executed remotely by the service platform. However, in certain scenarios, they may require the involvement of service engineers or customers (e.g., verifying that the scan of a phantom object does not produce any errors). Note that V can be an empty set as well, i.e., no verification is required.

A TMP recommendation operation 310 is configured to support a selection of a most suitable TMP to handle the current service case. The non-transitory computer readable medium 127 is configured to store detailed information regarding all third-party maintenance service providers that have an agreement with an Original Equipment Manufacturer (OEM; elsewhere herein referred to as the medical imaging device vendor). The detailed information regarding all third-party maintenance service providers forms the maintenance provider qualification data 130. The qualification data 130 includes detailed information regarding all TMPs that have an agreement with the OEM, including, for example, TMP performance history, TMP training history, or a combination thereof. In addition, a cost factor analysis can be performed. For example, two roughly equal qualified SEs would be resolved on availability and cost. However, the difference in qualification and cost can also be balanced within a selection algorithm. After the case creation operation 308, if it's preferred to involve a TMP for case handling, the TMP recommendation operation 310 includes selecting the most suitable service provider based on several criteria. The involvement of TMP in the case handling may occur in different phases. In some cases, the TMP is responsible for the entire case handling from remote diagnostic to onsite repair and reporting. In others, the TMP is selected only for onsite activities. The qualification of TMPs also differ. Some of them are qualified for end-to-end case handling while some are only qualified for onsite activities. Let a TMP be denoted by t. The set of all TMPs stored in the database is denoted by T. Given a service case c, for each TMP tᵢ in T, the recommender operation 310 calculates a score according to Equation 1:

$$score ( {t}_{i} , c ) = q ( {t}_{i} , c ) s ( {t}_{i} , c ) - {w}_{1} p ( {t}_{i} , c ) - {w}_{2} e ( {t}_{i} , c ) \qquad ( 1 )$$

The function q(tᵢ, c) determines if the TMP tᵢ is eligible for handling the case c where

$$q ( {t}_{i} , c ) = \{ \begin{matrix}1 , & {t}_{i} is qualified for handling c , \\ 0 , & {t}_{i} is not qualified for handling c\end{matrix} .$$

The function s(tᵢ, c) produces a score in the range of [0, 1], which indicates the likelihood of a successful case resolution by tᵢ. It can be determined by the expertise the TMP possesses and the historical case handling records on similar problem. The function p(tᵢ, c) produces a score in the range of (0, 1], which indicates the estimation of cost on handling the case c by tᵢ. w₁ is used to adjust the weight of the score for the overall score. The function e(tᵢ, c) produces a score in the range of (0, 1], which indicates the estimation of time required for handling the case c by tᵢ. w₂ is used to adjust the weight of the score for the overall score.

All available TMPs are ranked using the scoring function score(tᵢ, c). The recommender operation 310 can select the TMP automatically based on the ranking or provides a list of candidates to the domain expert who will make the final decision.

Once the most suitable TMP is selected, an information collector operation 312 uses rule-based logic to gather all relevant information that is passed to a security assessor operation 314. The information collector operation 312 queries data from available sources by using keywords to fetch device (log) data, metadata, remote diagnostics, service level agreement details, a configuration of the medical imaging device 120, other relevant service cases, etc. It also anonymizes and de-identifies data to make it untraceable.

The security assessor operation 314 includes assessing outputs from the information collector operation 312 to make sure no sensitive data is leaked to the TMP. The responsibility of managing privacy and access control lies on the vendor. This includes managing identities, authentication, authorization, and access control to provide privacy. As such, involving a TMP requires making sure all customer requirements regarding data sharing are in place. The security assessor operation 314 analyzes the collected information, which is ready to be shared with TMP, to avoid the risk of data breach. This is done by de-identifying and anonymizing data.

A maintenance service operation 316 includes performing the current service case (by the TMP), and once completed, the case is close and sent back to the vendor. Once the TMP has completed maintenance of the medical imaging device 120, it provides feedback to the vendor that the corresponding service case can be closed. Furthermore, a data exchange from the TMP to the vendor can be arranged. The vendor should learn as much as possible about customer issues and resolution approaches. For that, the TMP can record and send this data back to the vendor. The TMP wishes to minimize maintenance costs, hence minimize the parts ordered (often from the OEM). Upon a part order from the TMP to the vendor, the vendor can “verify” that the ordered parts make sense for a given issue. For example, when ordering a part, the TMP should also indicate the “error code” that it thinks justifies ordering the part. Advantageously, the vendor learns the association error-code/part and the TMP avoids ordering wrong parts.

A verification and case closure operation 318 includes verifying that the problem has been resolved and closes the service case. The successful closure of a service case may trigger the payment of the maintenance provider for the performed maintenance activities. When the vendor receives a trigger that maintenance has been completed by a TMP tᵢ for service case c, it first verifies that the maintenance is successful. For that purpose, it schedules the execution of all verification tests V, as specified in the service case. Maintenance is considered successful only if all tests pass, i.e., vᵢ(s)=1, ∀vᵢ∈V.

If a verification test fails, then the vendor decides how to continue with the maintenance of the medical system. In case the same TMP is selected to complete the maintenance, most of the information has been already provided, and the vendor only needs to decide which additional information to share with the TMP.

If all verification tests pass, then the vendor closes the service case c. This may trigger a payment process pay (t, c), that instructs a third-party billing system to compensate the TMP tᵢ for the performed activities.

A non-transitory storage medium includes any medium for storing or transmitting information in a form readable by a machine (e.g., a computer). For instance, a machine-readable medium includes read only memory (“ROM”), solid state drive (SSD), flash memory, or other electronic storage medium; a hard disk drive, RAID array, or other magnetic disk storage media; an optical disk or other optical storage media; or so forth.

The methods illustrated throughout the specification, may be implemented as instructions stored on a non-transitory storage medium and read and executed by a computer or other electronic processor.

The disclosure has been described with reference to the preferred embodiments. Modifications and alterations may occur to others upon reading and understanding the preceding detailed description. It is intended that the exemplary embodiment be construed as including all such modifications and alterations insofar as they come within the scope of the appended claims or the equivalents thereof.

1. A non-transitory computer readable medium storing:
data related to medical imaging devices generated from log files from the medical imaging devices;
instructions readable and executable by at least one electronic processor to:
retrieve, based on a query received for a current service case, log data from the log files of the medical imaging device for which the current service case is to be performed including at least operational data of the medical imaging device related to the current service case to be performed;
generate, from the retrieved log data, at least a subset of the operational data for the current service case to be provided to a maintenance provider; and
provide, on a display device of an electronic processing device operable by the maintenance provider, a user interface (UI) displaying information about the current service case.

2. The non-transitory computer readable medium of claim 1, further storing:
data related to qualifications of a plurality of maintenance providers; and wherein the instructions further include:
retrieve the qualifications of maintenance providers of the plurality of maintenance providers; and
select a maintenance provider from the plurality of maintenance providers to perform the current service case based on the retrieved log data and the retrieved qualifications.

3. The non-transitory computer readable medium of claim 2, wherein the data related to qualifications of the maintenance providers of the plurality of maintenance provider includes at least one of:
data related to one or more of an experience level of each maintenance provider, a likelihood of success of each maintenance provider in resolving the current service case, an availability of each maintenance provider, and a cost estimate for each maintenance provider.

4. The non-transitory computer readable medium of claim 1, wherein the instructions further include:
presenting, on the GUI of the display device of the electronic processing device operable by the maintenance provider, a dialog for ordering parts for the medical imaging device for which the current service case is to be performed.

5. The non-transitory computer readable medium of claim 1, wherein the at least one electronic processor is further programmed to generate, from the retrieved log data, at least a subset of the operational data for the current service case to be provided to a maintenance provider by:
anonymizing the log data from the log files of the medical imaging device for which the current service case is to be performed;
wherein the information about the current service case displayed on the UI includes the anonymized data.

6. The non-transitory computer readable medium of claim 1, wherein the at least one electronic processor is further programmed to generate, from the retrieved log data, at least a subset of the operational data for the current service case to be provided to a maintenance provider by:
filtering the log data from the log files of the medical imaging device for which the current service case is to be performed to remove proprietary information related to the medical imaging device; and
transferring the filtered data to the remote electronic processing device operable by the selected maintenance provider.

7. The non-transitory computer readable medium of claim 1, wherein the retrieved operational data includes one or more of control parameters for the medical imaging device, error codes generated by the medical imaging device, and sensor data generated by the medical imaging device.

8. The non-transitory computer readable medium of claim 1, wherein the at least one electronic processor is further programmed to:
apply a failure prediction model to the log data from the log files of the medical imaging device for which the current service case is to be performed to generate a failure prediction based on a subset of the log data; and
displaying, on an electronic processing device other than the electronic processing device operable by the selected maintenance provider, the failure prediction, and an identification of the subset of the log data on which the failure prediction is based;
wherein the information about the current service case displayed on the UI operable by the selected maintenance provider includes the failure prediction but does not include the identification of the subset of the log data on which the failure prediction is based.

9. The non-transitory computer readable medium of claim 1, wherein the retrieved log data includes one or more of alerts issued by one or more failure prediction models, log content generated proximate to a time of an alert, results of an automated root cause detect pattern run on the log data, a model of the medical imaging device, and configuration data of the medical imaging device.

10. The non-transitory computer readable medium of claim 1, further storing authorized parts lists for error codes and, in response to the selected maintenance provider ordering one or more parts for the medical imaging device for which the current service case is to be performed, the at least one electronic processor is further programmed to:
apply a verification filter comparing the ordered one or more parts with the authorized parts list for the associated error code for the current service case to determine whether the one or more parts are on the authorized parts list for the associated error code.

11. The non-transitory computer readable medium of claim 10, wherein the at least one electronic processor is further programmed to:
if the comparison indicates an ordered part is not on the authorized parts list for the associated error code, transmit a notification to a remote electronic processing device operable by a remote service engineer (RSE) for review by the RSE.

12. The non-transitory computer readable medium of claim 1, further storing authorized parts lists for error codes, and the at least one electronic processor is further programmed to:
apply a verification filter comparing one or more ordered parts with the authorized parts list for the associated error code for the current service case to one or more of a model of the medical imaging device, a configuration of the medical imaging device, and an identification of the medical imaging device.

13. The non-transitory computer readable medium of claim 1, wherein the at least one electronic processor is further programmed to:
receive a notification from the remote electronic processing device operable by the selected maintenance provider that the current service case has been completed;
perform one or more tests on the medical imaging device for which the current service case is to be performed to confirm that the current service case has been resolved.

14. The non-transitory computer readable medium, of claim 13, wherein the at least one electronic processor is further programmed to:
upon determining that the current service case has been resolved, initiate a payment to the selected maintenance provider.

15. A non-transitory computer readable medium, storing:
data related to medical imaging devices generated from log files from the medical imaging devices;
instructions readable and executable by at least one electronic processor to:
retrieve, based on a query received for a current service case, log data from the log files of the medical imaging device for which the current service case is to be performed including at least operational data of the medical imaging device related to the current service case to be performed;
generate, from the retrieved log data, at least a subset of the operational data for the current service case to be provided to a maintenance provider; and
provide, on a display device of an electronic processing device operable by the maintenance provider to perform the current service case, a user interface (UI) displaying information about the current service case and presenting a dialog for ordering parts for the medical imaging device for which the current service case is to be performed.

16. The non-transitory computer readable medium of claim 15, further storing:
data related to qualifications of a plurality of maintenance providers; and wherein the instructions further include:
retrieve the qualifications of maintenance providers of the plurality of maintenance providers;
select a maintenance provider from the plurality of maintenance providers to perform the current service case based on the retrieved log data and the retrieved qualifications.

17. The non-transitory computer readable medium of claim 15, wherein the at least one electronic processor is further programmed to generate, from the retrieved log data, at least a subset of the operational data for the current service case to be provided to a maintenance provider by:
anonymizing the log data from the log files of the medical imaging device for which the current service case is to be performed;
wherein the information about the current service case displayed on the UI includes the anonymized data.

18. The non-transitory computer readable medium of claim 16, wherein the at least one electronic processor is further programmed to generate, from the retrieved log data, at least a subset of the operational data for the current service case to be provided to a maintenance provider by:
filtering the log data from the log files of the medical imaging device for which the current service case is to be performed to remove proprietary information related to the medical imaging device; and
transferring the anonymized and filtered data to the remote electronic processing device operable by the selected maintenance provider.

19. The non-transitory computer readable medium of claim 15, wherein the at least one electronic processor is further programmed to:
apply a failure prediction model to the log data from the log files of the medical imaging device for which the current service case is to be performed to generate a failure prediction based on a subset of the log data; and
displaying, on an electronic processing device other than the electronic processing device operable by the selected maintenance provider, the failure prediction, and an identification of the subset of the log data on which the failure prediction is based;
wherein the information about the current service case displayed on the UI operable by the selected maintenance provider includes the failure prediction but does not include the identification of the subset of the log data on which the failure prediction is based.

20. A non-transitory computer readable medium storing:
data related to qualifications of a plurality of maintenance providers;
data related to medical imaging devices generated from log files from the medical imaging devices;
instructions readable and executable by at least one electronic processor to:
retrieve, based on a query received for a current service case, log data from the log files of the medical imaging device for which the current service case is to be performed including at least operational data of the medical imaging device related to the current service case to be performed;
generate, from the retrieved log data, at least a subset of the operational data for the current service case to be provided to a maintenance provider;
retrieve the qualifications of maintenance providers of the plurality of maintenance providers;
select a maintenance provider from the plurality of maintenance providers to perform the current service case based on the retrieved log data and the retrieved qualifications; and
provide, on a display device of an electronic processing device operable by the selected maintenance provider, a user interface (UI) displaying information about the current service case and presenting a dialog for ordering parts for the medical imaging device for which the current service case is to be performed.
